SEQUENCE LISTING (2 sequences)

SEQ ID NO: 1 - DNA, 25 bp, Artificial Sequence
  Description of Artificial Sequence Synthetic primer
cacatgttacttgaaggcctctgca

SEQ ID NO: 2 - DNA, 26 bp, Artificial Sequence
  Description of Artificial Sequence Synthetic primer
catgatgttatcagctccaagtcgta

(19) United States
(12) Patent Application Publication
(10) Pub. No.: US 2011/0124086 A1
(43) Pub. Date: May 26, 2011

(54) Hepatitis C Virus Culture System

(75) Inventors: Jinah Choi, Merced, CA (US); T. Jake Liang, Bethesda, MD (US)

(73) Assignees: The Regents of the University of California; National Institutes of Health

(21) Appl. No.: 12/895,518

(22) Filed: Sep. 30, 2010

Related U.S. Application Data
(60) Provisional application No. 61/248,226, filed on Oct. 2, 2009.
(60) Provisional application No. 61/385,477, filed on Sep. 22, 2010.

(51) Int. Cl. C12N 7/00; C12N 5/10; C12N 7/02

(52) U.S. Cl. 435/239; 435/325; 435/235.1

(57) ABSTRACT

This disclosure provides compositions and methods for producing infectious hepatitis C virus (HCV). The produced HCV can be infectious in vivo and in vitro. In one aspect, the disclosure provides an immortalized primary hepatocyte transformed with a DNA construct comprising a cDNA sequence of HCV.

### CROSS-REFERENCE TO RELATED APPLICATIONS

The present application claims priority under 35 U.S.C. §119(e) of U.S. Provisional Application Nos. 61/385,477 and 61/248,226, filed Sep. 22, 2010 and Oct. 2, 2009, respectively, the contents of each of which is hereby incorporated by reference into the present disclosure.

### BACKGROUND

Throughout this disclosure, various technical and patent publications are referenced to more fully describe the state of the art to which this disclosure pertains. These publications are incorporated by reference, in their entirety, into this application to more fully describe the state of the art to which this disclosure pertains.

Hepatitis C virus (HCV) is an etiologic agent of severe liver diseases affecting humans, including cirrhosis and hepatocellular carcinoma. HCV is estimated to infect about 170 million people worldwide, including four million in the U.S. Hepatocytes in the liver are the primary sites of HCV replication. HCV only infects humans and high level primates (chimpanzees), which poses significant challenge to studying this virus. Recently, chimeric mouse with humanized liver has been developed that can be infected with HCV. Nevertheless, a lack of a convenient cell culture system that efficiently replicates infectious HCV severely hampered basic research and drug screening. HCV replicons, which are artificial constructs that genetically modify the HCV genome to allow the replication of HCV RNA genome at a high level in Huh7 human hepatoma cells, have proven useful for studying viral RNA replication and translation and for screening for antiviral drugs that target these steps of the viral replication cycle. However, as these replicons do not generate infectious HCV in cell culture, these systems do not fully recapitulate the complete replication cycle of this virus.

Con1 replicon and other replicons are useful for studying viral RNA replication/translation and certain aspects of HCV biology, and can be used to screen for antiviral drugs that target the RNA replication/translation steps of the viral replication cycle. However, these do not generate infectious HCV in cell culture and therefore, do not fully recapitulate complete replication process of the virus.

Recently, a cell culture system that supports the complete replication of hepatitis C virus (HCV) of genotype 2a was discovered. However, this cell culture system does not support replication of HCV genotype 1, the most prevalent HCV genotype. Even in the event that the HCV is replicated in a tissue culture system, the produced HCV may not be infectious. For example, Con1 replicons of HCV genotype 1 b support viral RNA genome replication but do not generate infectious HCV in cell culture. Further, Hutchinson strain of genotype 1 a shows limited infection efficiency and thus not widely used (Yi et al. (2006) Proc. Natl. Acad. Sci. USA 103:2310-2315 and Yi et al. (2009) Methods Mol. Biol. 510:337-346).

Huh7 or Huh7.5.1 cells have been used to generate genotype 1 b HCV (Heller et al. (2005) Proc. Nat. Acad. Sci. USA 102(7):2579-83). Cell culture medium derived from HCV genotype 1 b transfected Huh7 or Huh7.5.1 cells established productive infection in chimpanzees. However, there is no evidence that the produced HCV can infect cells in vitro, limiting its use in basic research as well as drug screening.

### SUMMARY OF THE INVENTION

The present disclosure provides, in one embodiment, a cell culture system and method that generate infectious HCV. In one aspect, the HCV is of genotype 1a (Hutchinson strain), 1b or 2. In another aspect, the HCV is infectious in vitro.

It is herein discovered that the use of telomerase-reconstituted (immortalized) primary human fetal hepatocytes can be used as the host to generate infectious HCV of genotype 1b in cell culture. The telomerase-reconstituted primary human fetal hepatocytes (e.g., FH-CG1 bRbz cells) can be stably transfected with pEF-CG1 bRbz/Neo construct. FH-CG1 bRbz cells generated infectious HCV of genotype 1a, 1b or 2, which in turn can be used to infect hepatocytes in vitro. After inoculating naïve Huh7 cells with cell culture medium from FH-CG1 bRbz cells, HCV RNA and HCV proteins were detected in the infected cells. The HCV RNA decreased with interferon alpha in the infected cell. The present disclosure demonstrates that FH-CG1 bRbz cells continued to produce infectious HCV for at least up to 32 cell passages.

This is unexpected in view of the recent efforts by researchers that tried to enhance the infectivity of cell culture-generated HCV by treating cells with an inhibitor that is likely to alter the normal replication cycle of the virus. Primary hepatocytes-based systems that generate inter-genotypic JFH1 or several HCV genotypes are being described in publications published in 2010 (Banaudha et al. (2010) Hepatology 51(6):1922-32; Podevin et al. (2010) Gastroenterology, 2010 Jul. 2. [Epub ahead of print]; and Jones et al. (2010) Nat. Biotechnol, 28(2):167-171). Still, these systems require complex cell culture methods, require artificial methods such as co-incubation with a lipofection solution, or are limited by the finite lifespan of the primary cells and/or low infectivity.

Further, the culturing systems and methods of the present disclosure can provide a convenient in vitro system for generating sufficient infectious HCV particles, for studying complete genotype 1b HCV replication in cell culture and for testing antivirals in the context of complete viral replication cycle for this viral genotype. These systems are easy to maintain, do not require complex cell culture technique, and behave as a continuous cell line consistently producing infectious HCV.

The systems and methods of the present disclosure also are useful for studying complete replication of HCV in vitro and for generating sufficient infectious HCV particles for performing in vitro HCV infectivity studies without complicated cell culture or artificial procedures. This allows one to test antivirals in vitro, in the context of complete viral replication cycle for this genotype. The FH-CG1b system potentially provides a convenient in vitro system for generating sufficient level of infectious HCV particles for studying complete replication cycle of HCV, including genotype 1b HCV. Genotype 1 is the most prevalent viral genotypes in the U.S. and shows the highest level of resistance to available anti-HCV therapy.

Accordingly, this disclosure provides compositions and methods for producing infectious hepatitis C virus (HCV). In one aspect, the disclosure provides an immortalized, primary hepatocyte transformed with a DNA construct comprising a cDNA sequence of hepatitis C virus (HCV). In some embodiments, the HCV is HCV genotype 1, such as but not limited to 1a or 1b, or genotype 2. In some embodiments, the DNA construct is CG1 bRBz.

In some embodiments, the immortalized primary hepatocyte is a telomerase reconstituted immortalized mammalian primary hepatocyte, or alternatively, a telomerase reconstituted immortalized human primary hepatocyte.

Also provided is an isolated primary hepatocyte as described herein which, in a further aspect, can be a substantially homogeneous culture of the hepatocytes of any of the above embodiments.

The disclosure further provides a method for producing an infectious hepatitis C virus (HCV) comprising, or alternatively consisting essentially of, or alternatively consisting of, transforming an immortalized primary hepatocyte with a DNA construct comprising a cDNA of the HCV thereby producing an infectious HCV. In some embodiments, the immortalized primary hepatocyte is a telomerase reconstituted immortalized primary hepatocyte.

### BRIEF DESCRIPTION OF THE FIGURES

FIG. 1 shows that HCV genotype 1 b (CG1b) produced by the culture system of this disclosure infected Huh7 cells in vitro. JFH1 replicons of HCV 2a was used as positive control.

FIG. 2 shows that HCV genotype 1 b (CG1b) produced by the culture system of this disclosure infected Huh7.5.1 cells in vitro. JFH1 replicons of HCV 2a was used as positive control.

FIG. 3 shows that telomerase-reconstituted primary human hepatocytes, stably transfected with CG1 bRbz or control constructs (pEF vector only or GND) were analyzed for (+/−) and (−) strand HCV RNAs by qRT-PCR. The +RT/−RT ratio denotes approximate ratio of HCV RNA signal in the presence and absence of reverse transcriptase.

FIG. 4 shows infectivity of CG1 bRbz medium from transfected primary human hepatocytes. (A) HCV in the cell culture medium from CG1 bRbz, GNDRbz, and pEF control hepatocyte clones were concentrated as described in Methods and used to inoculate Huh7 or Huh7.5 cells. Then, HCV RNA level was determined by qRT-PCR at indicated time points. Medium from JFH1 RNA-transfected Huh7 cells served as positive control for infectivity. Note that with later passage medium, GNDRbz-inoculated cells were found to contain HCV RNA with GDD sequence by RT-PCR sequencing. GND and GDD here refer to single letter amino acid abbreviations, GND (glycine-asparagine-aspartatic acid; replication-defective mutant) and GDD (glycine-aspartatic acid-aspartatic acid; replication-competent wildtype virus sequence). (B) Huh7 cells inoculated with later passage FH cell culture medium were also analyzed for HCV core and E2 proteins by immunofluorescence/confocal microscopy. Image was acquired at 40× and at the gain of 8.6 for Core and 8.7 for E2. pEF-inoculated cells were also imaged at a much higher gain (10.0) to show that cells were present in these panels.

FIG. 5 shows antiviral activity of IFNα. Huh7 cells were infected with hepatocyte-derived medium. Then, after 48 hrs, cells were treated with 0 or 100 U/ml of IFNα once and analyzed for HCV RNA by qRT-PCR after 24 hrs. N.D. indicates not detectable or below the level of detection.

FIG. 6 shows the effect PKR and Jak2 inhibitor on HCV. Cg1bRbz cells were treated with C5 (1 μM) or Jak2 inhibitor II (10 and 50 μM) for 48 or 96 hrs and analyzed for intracellular HCV RNAs by qRT-PCR. Huh7 cells transfected with JFH1 RNA were also used as a control. Bottom right—Huh7 cells were inoculated with virus samples prepared from control and Jak2 inhibitor II-treated later passage FH cell culture medium and analyzed for HCV RNA after 48 hrs by qRT-PCR.

### DETAILED DESCRIPTION OF THE INVENTION

### Definitions

The practice of the present disclosure will employ, unless otherwise indicated, conventional techniques of tissue culture, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, A Laboratory Manual; and Animal Cell Culture (R. I. Freshney, ed. (1987)).

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure.

A “composition” is intended to mean a combination of active agent, cell or population of cells and another compound or composition, inert (for example, a detectable agent or label or biocompatible scaffold) or active, such as a growth and/or differentiation factor.

An “effective amount” is an amount sufficient to effect beneficial or desired results. For the purpose of this application, an effective amount of telomerase reconstituted primary hepatocytes refers to an amount that is sufficient to produce a desired amount of infectious HCV. An effective amount of DNA construct refers to an amount that is sufficient to effectively transform the telomerase reconstituted primary hepatocytes.

The term “isolated” as used herein refers to molecules or biological or cellular materials being substantially free from other materials, e.g., greater than 70%, or 80%, or 85%, or 90%, or 95%, or 98%. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source and which allow the manipulation of the material to achieve results not achievable when present in its native or natural state, e.g., recombinant replication or manipulation by mutation. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides, e.g., with a purity greater than 70%, or 80%, or 85%, or 90%, or 95%, or 98%. The term “isolated” is also used herein to refer to cells or tissues that are isolated from other cells or tissues and is meant to encompass both cultured and engineered cells or tissues.

A “recombinant” nucleic acid, polypeptide, protein or cell, refers an artificial combination that is created by combining material not naturally occur together. In one embodiment, it is created through the introduction of relevant DNA into an existing organismal DNA, such as the plasmids of bacteria, to code for or alter different traits for a specific purpose, such as antibiotic resistance. A “recombinant” can also refer to a polypeptide that is derived from a recombinant nucleic acid.

The terms “nucleic acid”, “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, small interference RNA (siRNA), double strand RNA (dsRNA), ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

A “ribozyme”, from ribonucleic acid enzyme, also called RNA enzyme or catalytic RNA, refers to an RNA molecule that catalyzes a chemical reaction. Many natural ribozymes catalyze either the hydrolysis of one of their own phosphodiester bonds, or the hydrolysis of bonds in other RNAs, but they have also been found to catalyze the aminotransferase activity of the ribosome. A non-limiting example of a ribozyme that functions in hepatocytes is described in Benedict et al. (1998) Carcinogenesis 19:1223-30.

“Immortalized” cells refer to cells that are not limited by the Hayflick limit (where cells no longer divide because of DNA damage or shortened telomeres). The term immortalization was first applied to cancer cells that expressed the telomere-lengthening enzyme telomerase, and thereby avoided apoptosis (programmed cell death). Among the most commonly used cell lines are HeLa and Jurkat, both of which are immortalized cancer cell lines. Normal stem cells and germ cells can also be said to be immortal (when referring to the cell line). Normal somatic cells can also be immortalized by methods including, but not limited to, telomerase reconstitution (see, e.g., Wege et al. (2003) Gastroenterology 124:432-44).

“Immortalized primary hepatocyte” refers to a primary hepatocyte that is immortalized. Unlike immortalized liver carcinoma cell lines such as HepG2, an immortalized primary hepatocyte is non-transformative and non-tumorigenic. A non-limiting example of an immortalized primary hepatocyte is a telomerase reconstituted primary hepatocyte, as previously described in Wege et al. (2003) Gastroenterology 124:432-44.

### Hepatitis C Viruses and DNA Constructs

“Hepatitis C virus” or “HCV” is a small (55-65 nm in size), enveloped, positive sense single strand RNA virus in the family Flaviviridae. RNA viruses carry their genetic information in the form of RNA; however, once the virus infects a cell, the RNA is reverse-transcribed into the DNA form which integrates into the genomic DNA of the infected cell. The integrated DNA form is called a provirus.

Hepatitis C virus has a positive sense RNA genome that consists of a single open reading frame of 9600 nucleoside bases. At the 5′ and 3′ ends of the RNA are the UTR regions, that are not translated into proteins but are important to translation and replication of the viral RNA. The 5′ UTR has a ribosome binding site that starts the translation of a 3000 amino acid containing protein that is later cut by cellular and viral proteases into 10 active structural and non-structural smaller proteins.

Based on genetic differences between HCV isolates, the hepatitis C virus species is classified into six genotypes (1-6) with several subtypes within each genotype (represented by letters). Subtypes are further broken down into quasispecies based on their genetic diversity. The preponderance and distribution of HCV genotypes varies globally. For example, in North America, genotype 1a predominates followed by 1b, 2a, 2b, and 3a. In Europe, genotype 1 b is predominant followed by 2a, 2b, 2c, and 3a. Genotypes 4 and 5 are found almost exclusively in Africa.

Replication of HCV involves several steps. HCV has a high rate of replication with approximately one trillion particles produced each day in an infected individual. Due to lack of proofreading by the HCV RNA polymerase, HCV also has an exceptionally high mutation rate, a factor that may help it elude the host's immune response.

HCV mainly replicates within hepatocytes in the liver. Once inside the hepatocyte, HCV initiates the lytic cycle. It utilizes the intracellular machinery necessary to accomplish its own replication. RNA replication takes places via the viral RNA-dependent RNA polymerase NS5B, which produces a negative-strand RNA intermediate. The negative strand RNA then serves as a template for the production of new positive-strand viral genomes. New virus particles are thought to bud into the secretory pathway and are released at the cell surface.

DNA constructs can be used to produce HCV particles. Such a DNA construct can be composed of a plasmid or viral vectors connected to the cDNA of HCV. The DNA construct can further comprise one or more ribozyme sequences to facilitate effective production. A non-limiting example of a DNA construct including a HCV genotype 1 b cDNA is CG1 bRBz as previously described in Heller et al. (2005) Proc. Nat. Acad. Sci. USA 102(7):2579-83.

### MODES FOR CARRYING OUT THE INVENTION

In one aspect, the disclosure provides an immortalized primary hepatocyte transformed with a DNA construct comprising a cDNA sequence of hepatitis C virus (HCV). In one aspect, the hepatocyte is isolated or recombinant.

Also provided is a substantially homogeneous culture of the hepatocytes of any of the embodiments as described herein.

The disclosure further provides methods for producing infectious HCV with the disclosed culturing system. One aspect of the disclosure provides a method for producing an infectious hepatitis C virus (HCV) comprising, or alternatively consisting essentially of, or yet further consisting of, transforming an immortalized primary hepatocyte with a DNA construct comprising, or alternatively consisting essentially of, or yet further consisting of, a cDNA of the HCV thereby producing an infectious HCV. In a further aspect the cell is cultured under conditions that allow for reproduction of infectious HCV. In a further aspect, the method further comprises, or alternatively consists essentially of, or yet further consist of, separating or isolating the infectious HCV from the cell or the cell culture system or media.

In some embodiments, the immortalized primary hepatocyte is a primary hepatocyte immortalized by telomerase reconstitution. In some embodiments, the hepatocyte is a mammalian hepatocyte. In each of these embodiments, the hepatocyte can be isolated. Non-limiting examples of mammal include simian, bovine, porcine, murine, rats, and human.

In one aspect of any of the above embodiments of the disclosure, the DNA construct further comprises at least one ribozyme, or alternatively at least two ribozymes. When there are two ribozymes in the DNA construct, in some embodiments, the cDNA sequence is located between the ribozymes.

In another aspect of any of the above embodiments of the disclosure, the HCV is HCV genotype 1 or 2. Non-limiting examples of HCV genotype 1 includes genotypes 1a and 1b. In one aspect, the HCV is HCV 1b. In a particular aspect, the DNA construct is CG1 bRBz as previously described in Heller et al. (2005) Proc. Nat. Acad. Sci. USA 102(7):2579-83.

The disclosure, in another aspect, provides an infectious HCV produced by any embodiment of the method of the disclosure. Also provided is a method to prepare such be isolating them from the culture system.

Also provided is a kit for use in producing an infectious hepatitis C virus (HCV) comprising, or alternatively consisting essentially of, or consisting of, a DNA construct comprising a cDNA of the HCV and instructions for use. The kits can be used to screen candidate agents such as biologics or small molecules that can inhibit HCV replication and/or infectivity. The kits can further contain a telomerase reconstituted immortalized primary hepatocyte or instructions for obtaining and culturing them. In one aspect, the DNA construct is transformed into the hepatocyte.

The following examples are provide to illustrate select embodiments of the disclosure as disclosed and claimed herein.

### Experimental Examples

### Example 1

### HCV Constructs

pEF-CG1 bRbz/Neo is a DNA construct including the cDNA sequence of HCV genotype 1 b, as described in Heller et al. (2005) Proc. Nat. Acad. Sci. USA 102(7):2579-83. pEF-CG1 bRbz/Neo and replicative-null pEF-CG1b GNDRbz/Neo were obtained from Dr. T. Jake Liang of the National Institutes of Health, and were used to study genotype 1b HCV replication (Heller et al. (2005) Proc. Nat. Acad. Sci. USA 102(7):2579-83, Kato et al. (2007) J. Virol. 81:4405-11). pJFH1, kindly provided by Dr. Takaji Wakita, which generates infectious virus particles of genotype 2a, its replicative-null mutant (pJFH1-GND), and subgenomic pSgJFH1-Luc, which supports viral RNA genome replication without generating virus particles, were used for comparison (Wakita et al. (2005) Nat. Med. 11:791-6, Kato et al. (2005) J. Virol. 79:592-6).

### Cells, Transfection/Infection, and Tissues

Telomerase-reconstituted primary human hepatocytes were described previously (Wege et al. (2003) Gastroenterology 124:432-44) and were kindly provided by Dr. Mark Zern of University of California, Davis. Telomerase-reconstituted primary human hepatocytes were transfected with pEF-CG1 bRbz/Neo, pEFCG1 bGNDRbz/Neo, or control EF-driven vector alone as described for Huh7 (Choi et al. (2004) Hepatology 39:91-9). Briefly, 5×10⁶ cells were mixed with 10 μg JFH1 RNA or pEF plasmids in 0.4 ml of Opti-MEM (Invitrogen) and electroporated at 260 V and 950 pF. Then, the cells were maintained in Dulbecco's Modified Eagle Medium supplemented with 10% fetal bovine serum and penicillin plus streptomycin (Invitrogen). For CG1b transfections, cells that are stably transfected with these constructs were selected and maintained in G418-containing medium (Invitrogen). Telomerase-reconstituted primary human hepatocytes, derived from human fetal hepatocytes, exhibit normal hepatocyte functions and can be passaged up to 300 times without losing hepatocyte characteristics (Wege et al. (2003) Gastroenterology 124:432-44). These hepatocytes were maintained in Dulbecco's Modified Eagle Medium, containing 10% heat-inactivated fetal bovine serum, 4 mM Lglutamine, 5 μg/ml insulin, 2.4 μg/ml hydrocortisone (Sigma Aldrich), and penicillin plus streptomycin (Invitrogen) (Wege et al. (2003) Gastroenterology 124:432-44). For virus infection, 2 ml of the extracellular medium from JFH1-transfected cells was used to inoculate naïve Huh7 cells with 3 ml of fresh medium, as described (Wakita et al. (2005) Nat. Med. 11:791-6, Kato et al. (2005) J. Virol. 79:592-6). Then, the cells were cultured and harvested at various time points.

### Results

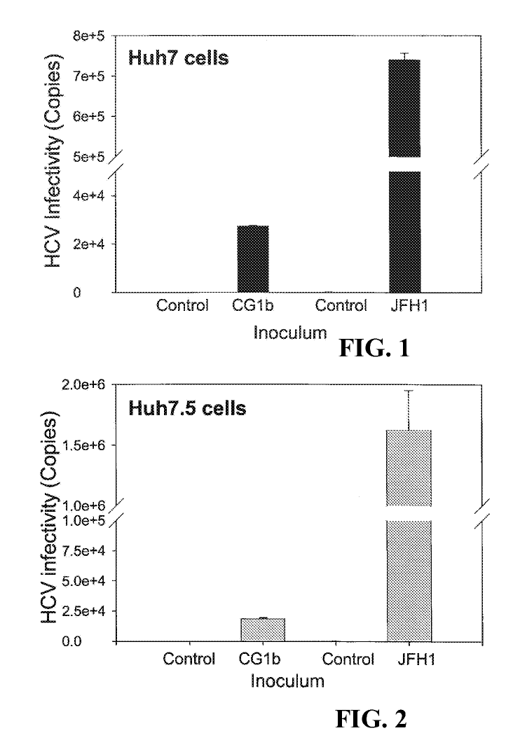

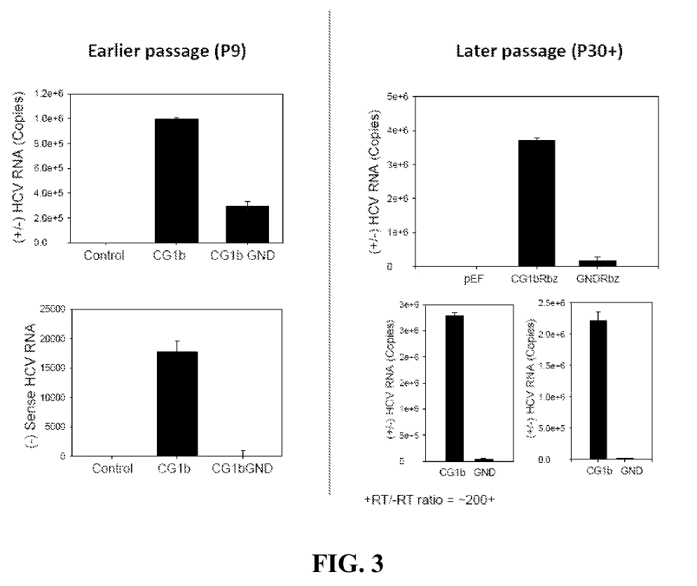

The telomerase reconstituted primary human fetal hepatocytes were stably transfected with pEF-CG1bRbz/Neo construct. After inoculating naive Huh7 or Huh7.5.1 cells with cell culture medium from FH-CG1bRbz transfected cells, both positive-sense and negative-sense HCV RNA were detected in the Huh7 and Huh7.5.1 cells. As shown in FIG. 1, HCV genotype 1b (CG1b) produced by the culture system of the disclosure infected Huh7 cells in vitro. JFH1 replicons of HCV 2a was used as positive control. As shown in FIG. 2, HCV genotype 1b (CG1b) produced by the culture system of the disclosure infected Huh7.5.1 cells in vitro. JFH1 replicons of HCV 2a was used as positive control.

### Example 2

This example shows a continuous cell culture system that generates infectious HCV of genotype 1 b. By using telomerase-reconstituted (immortalized) primary human fetal hepatocytes that can be passaged up to 300 times without losing hepatocyte characteristics, these cells, when stably transfected with CG1 bRbz construct, generate infectious HCV of genotype 1 b, capable of infecting human hepatocytes in vitro.

### Methods

### HCV Constructs

pJFH1, which generates infectious virus particles of genotype 2a, its replicative-null mutant (pJFH1-GND) and genotype 1b pEF-CG1bRbz/Neo plus its replicative-null mutant (pEFCG1b GNDRbz/Neo) were used (Wakita et al. (2005) Nat. Med. 11:791-6; Heller et al. (2005) Proc. Natl. Acad. Sci. USA 102:2579-83).

### Cell Culture and HCV Transfection/Infection

Huh7 human hepatoma cells were transfected with in vitro-transcribed JFH1 RNA and cultured, as previously described (Choi et al. (2004) Hepatology 39:91-9). Telomerase-reconstituted primary human hepatocytes were transfected with pEF-CG1 bRbz/Neo, pEF-CG1 bGNDRbz/Neo, or control EF-driven vector alone as described for Huh7, and cell clones that are stably transfected with these constructs were selected and maintained in G418-containing medium (Invitrogen) (Wege et al. (2004) Gastroenterology 124:432-44). For virus infection, the extracellular medium from JFH1-transfected cells and CG1b transfected cell clones was cleared by low speed centrifugation at 5,000 rpm for 10 min and concentrated ˜10 fold by ultracentrifugation at 21,000 rpm for 6.5 hrs at 4° C. The medium was also pre-treated with RNase (and also with DNase for CG1b medium) for 30 min at room temperature prior to these centrifugation steps. Then, the concentrated virus samples were used to inoculate naïve Huh7 or Huh7.5 cells (Wakita et al. (2005) Nat. Med. 11:791-6). Then, the cells were cultured and harvested at various time points, as indicated in the section below.

### HCV Quantitation

HCV RNA was quantified by quantitative reverse transcriptase polymerase chain reaction (qRT-PCR) as described. Intracellular HCV RNA levels were normalized by glyceraldehydes 3-phosphate dehydrogenase mRNA content. Standard curves were generated using in vitro-transcribed HCV RNA's. Control reactions containing no reverse transcriptase were also carried out as controls. HCV protein levels were visualized by immunofluorescence staining followed by confocal microscopy. Briefly, cells were fixed with 3.5% formaldehyde for 5 min and incubated with phosphate buffered saline, containing 1%(w/v) bovine serum albumin, 0.05 (w/v) % NaN₃, and 0.02 (w/v) % saponin. Samples were subsequently incubated with core and E2 antibodies and then with fluorophore-conjugated secondary antibodies, mounted on microscope slides, and imaged via confocal laser scanning microscopy (C1, Nikon).

### RT-PCR Sequencing

The NS5B GDD region (nt. 8547-8555 of CG1b sequence) was reverse transcribed with AMV reverse transcriptase (Promega Corp.) and the cDNA, amplified with Pfu DNA polymerase (Stratagene/Agilant). Random primers were used for reverse transcription. Primer sequences for polymerase chain reaction were 5′-CACATGTTACTTGAAGGCCTCTGCA-3′ (forward, SEQ ID NO: 1) and 5′-CATGATGTTATCAGCTCCAAGTCGTA-3′ (reverse, SEQ ID NO: 2). The 219-mer cDNA product was then eluted from polyacrylamide gel, ethanol precipitated, and submitted for sequencing at the University of California Berkeley Sequencing (Berkeley, Calif.). Sequences were aligned against CG1b sequence, using Sequence Scanner v1.0 (Applied Biosystems) and GeneRunner software.

### Statistics

Data were analyzed using SigmaStat 3.1 (Jandel Scientific). A p value 0.05 was considered significant. Data are presented as means ±standard error of the mean.

### Results

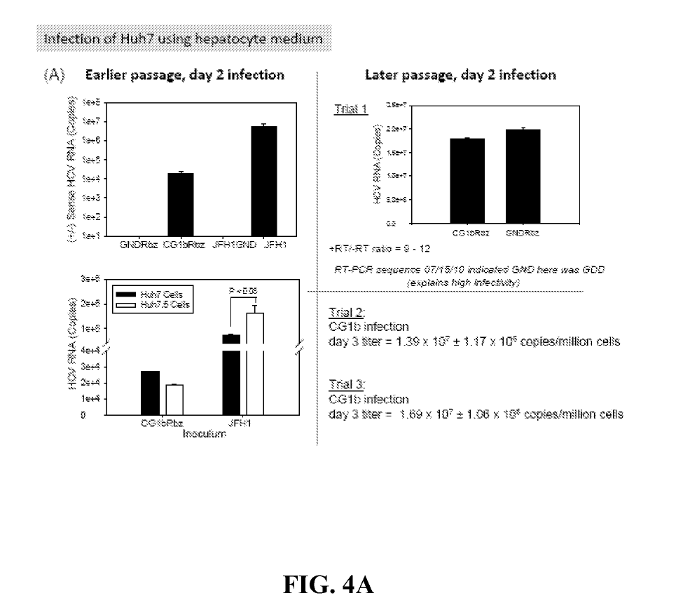

Telomerase-reconstituted primary human hepatocytes (abbreviated as FH indicating “fetal hepatocytes”), derived from human fetal hepatocytes, exhibit normal hepatocyte functions and can be passaged up to 300 times in cell culture without losing hepatocyte characteristics. These cells were transfected with pEF-CG1 bRbz/Neo, replicative-null pEF-CG1 bGNDRbz/Neo, or control EF-driven vector alone as described (Wakita et al. (2005) Nat Med 11:791-6; Heller et al. (2005) Proc. Natl. Acad. Sci. USA 102:2579-83). To increase the viral titer, cell clones were selected that are resistant to G418. As G418 normally kills these cells, only cells that are transfected with above constructs and contain the G418-resistance gene would survive. Both positive and negative sense HCV RNAs could be demonstrated in the G418-selected FHCG1 bRbz cells (stably transfected with CG1 bRbz/Neo construct) by quantitative reverse transcription polymerase chain reaction (qRT-PCR) (FIG. 3). Control cells, stably transfected with empty vector alone, did not show HCV RNA, as expected. GNDRbz produces positive sense HCV mRNA by host transcription but no (−) sense viral RNA is generated due to a critical mutation (GDD:GND) in the viral replicase gene. As such, FH-CG1 bGNDRbz showed HCV RNA but no (−) strand HCV RNA (FIG. 3). HCV core protein could also be detected readily in the CG1 bRbz cells. HCV RNA persisted for at least ˜30 passages.

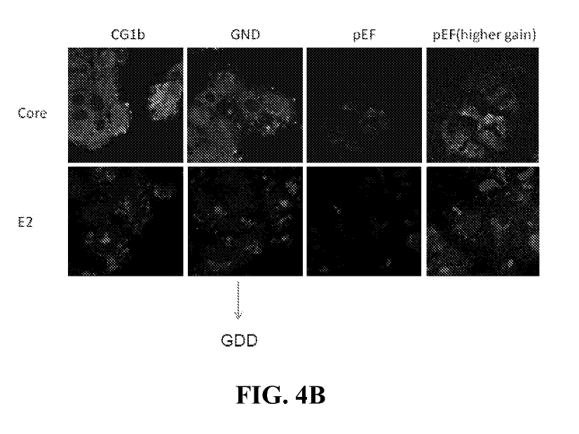

To examine whether these cells generated infectious virus particles, naïve Huh7 and Huh7.5 human hepatoma cells were incubated with medium collected from FH-CG1 bRbz cells (FIG. 4). After 48 hrs, these cells were analyzed for HCV RNA by qRT-PCR. Medium from vector only-transfected control hepatocytes and FH-CG1 bGNDRbz cells served as negative controls. Medium from JFH1 RNA-transfected Huh7 cells, which generate infectious genotype 2a HCV, were used as a positive control. HCV RNA was detected in Huh7 and Huh7.5 cells infected with medium from FH-CG1 bRbz cells as well as JFH1 (FIG. 4A). In contrast, the RNA signal was absent or remained near the detection limit of these qPCR measurements in the control pEF and GNDRbz-inoculated cells (FIG. 4A). With later FH passage numbers, the HCV RNA titer in the infected Huh7 cells rose from ˜10⁴ copies to 10⁷ copies/million cells, which is comparable to the infectivity of JFH1 (FIG. 4). HCV core and E2 proteins could also be detected in CG1 bRbz-infected Huh7 cells (FIG. 4B). Medium from FHpEF cells served as negative control.

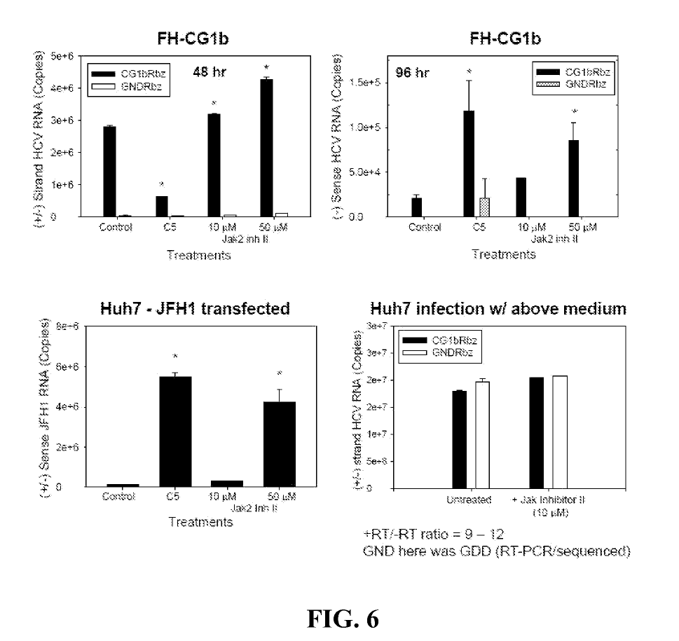

Interestingly, at late FH passage numbers, GNDRbz-infected Huh7 exhibited significant levels of HCV RNA and protein levels as the CG1 bRbz-infected cells. The HCV RNA from GNDRbz cells, therefore, was reverse transcribed, amplified by polymerase chain reaction, and sequenced. Sequencing results indicated that the HCV RNA in these cells have GGA GAC GAC sequence corresponding to GDD instead of GND sequence, which explains high HCV signals in these cells (FIG. 4A; also, see FIG. 6).

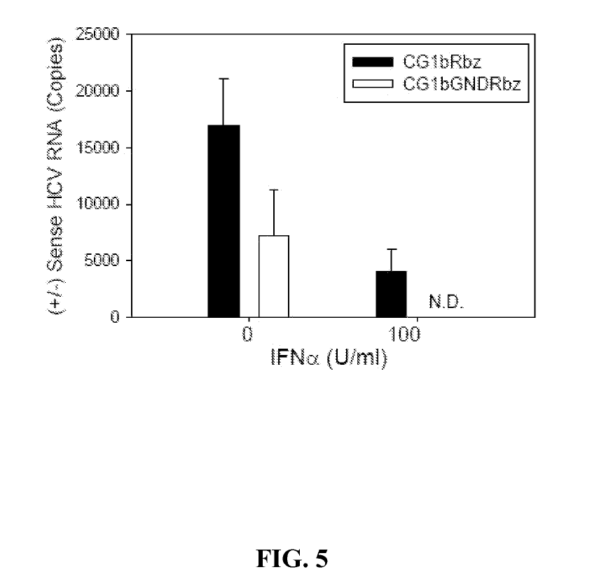

To confirm that the CG1b-infected cells exhibited HCV RNA replication, Huh7 cells inoculated with medium from FH-CG1 bRbz cells were challenged with interferon alpha (IFNα; 100 U/ml) for 24 hrs. Then, whether the viral titer decreased with this treatment was examined. IFNα decreased HCV titer in the CG1b-infected Huh7 cells as expected (FIG. 5). The data indicate that the FH system is producing infectious HCV of genotype 1b that can infect Huh7 cells in culture.

It was then examined whether suppressing the hepatocyte innate immunity by inhibiting Jak/Stat pathway and/or PKR could increase the HCV titer in Applicants' FH cell clones and therefore, increase the infectivity of CG1b. HCV RNA level was analyzed in FH-CG1 bRbz cells after 48 or 96 hr incubation with C5, an inhibitor of PKR, or Jak2 Inhibitor II. As shown in FIG. 6, intracellular HCV RNA increased with these inhibitors. Huh7 cells transfected with JFH1 RNA also showed an increase in the intracellular HCV RNA with these treatments (FIG. 6). The HCV infectivity increased only modestly, however, using medium from Jak2 inhibited FH-CG1 bRbz cells compared to medium from control FH-CG1bRbz cells (FIG. 6).

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. An isolated or recombinant immortalized primary hepatocyte transformed with a DNA construct comprising a cDNA sequence of hepatitis C virus (HCV).

2. The hepatocyte of claim 1, wherein the DNA construct further comprises at least one ribozyme.

3. The hepatocyte of claim 1, wherein the DNA construct comprises at least two ribozymes.

4. The hepatocyte of claim 3, wherein the cDNA sequence of the HCV is located between the ribozymes.

5. The hepatocyte of claim 1, wherein the HCV is HCV genotype 1 or 2.

6. The hepatocyte of claim 1, wherein the HCV is HCV genotype 1a or 1b.

7. The hepatocyte of claim 1, wherein the HCV is HCV genotype 1 b.

8. The hepatocyte of claim 1, wherein the DNA construct is CG1 bRBz.

9. The hepatocyte of claim 1, wherein the hepatocyte is a telomerase reconstituted immortalized mammalian primary hepatocyte.

10. The hepatocyte of claim 1, wherein the hepatocyte is a telomerase reconstituted immortalized human primary hepatocyte.

11. A substantially homogeneous culture of the hepatocytes of claim 1.

12. A method for producing an infectious hepatitis C virus (HCV) comprising transforming an isolated immortalized primary hepatocyte with a DNA construct comprising a cDNA sequence of the HCV thereby producing an infectious HCV.

13. The method of claim 12, further comprising culturing the hepatocyte under conditions that promote production of infectious HCV.

14. The method of claim 12, further comprising isolating the infectious HCV from the hepatocyte or cell culture.

15. The method of claim 12, wherein the DNA construct further comprises at least one ribozyme.

16. The method of claim 12, wherein the DNA construct comprises at least two ribozymes.

17. The method of claim 14, wherein the cDNA sequence of the HCV is located between the ribozymes.

18. The method of claim 12, wherein the HCV is HCV genotype 1 or 2.

19. The method of claim 12, wherein the HCV is HCV genotype 1a or 1b.

20. The method of claim 12, wherein the HCV is HCV genotype 1 b.

21. The method of claim 12, wherein the DNA construct is CG1 bRBz.

22. The method of claim 12, wherein the hepatocyte is a telomerase reconstituted immortalized mammalian primary hepatocyte.

23. The method of claim 12, wherein the hepatocyte is a telomerase reconstituted immortalized human primary hepatocyte.

24. An isolated infectious HCV produced by the method of claim 12.
